(19) United States
(12) Patent Application Publication
(10) Pub. No.: US 2025/0364335 A1
(43) Pub. Date: Nov. 27, 2025

(54) SYSTEMS AND METHODS FOR WAFER PROCESSING WITH SENSOR TECHNOLOGIES

(71) Applicant: Tokyo Electron Limited, Tokyo (JP)

(72) Inventors: Michael Carcasi, Leuven (BE); Ihsan Simms, Austin, TX (US); Rencheng Dong, Austin, TX (US); Derek William Bassett, Austin, TX (US); Sean Berglund, Austin, TX (US)

(21) Appl. No.: 18/671,015

(22) Filed: May 22, 2024

(51) Int. Cl. H01L 21/66; G01N 33/00; H01L 21/67

(52) U.S. Cl. CPC H01L 22/26; G01N 33/0006; H01L 21/67253

(57) ABSTRACT

A method of processing a substrate includes loading and processing the substrate in a process chamber, and obtaining first gas sensor data generated by interaction of a first target gas with a first gas sensor fluidly coupled to a headspace of the process chamber. The method includes determining a first metric for the processing based on the first gas sensor data, the determining including comparing the first gas sensor data with a first gas calibration data set. The method includes terminating the processing of the substrate based on the first metric for the processing.

### TECHNICAL FIELD

The present invention relates generally to methods for wafer processing, and, in particular embodiments, to systems and methods for wafer processing with sensor technologies.

### BACKGROUND

Manufacturing of semiconductor wafers generally involves a sequence of processing steps, such as patterning with photoresist, photoresist development, wet or dry etching, thermal annealing, cleaning, drying, etc. These steps may require treating the wafer surface with one or more process chemicals. The process chemicals, their residues, fragments of uniform or patterned layers that have been stripped or etched, and particulates from the surface may all be rinsed away or otherwise removed after each step. Even the rinsing liquids must ultimately be removed, and in some cases volatile organic compounds (VOCs) and other species (such as water) may be driven out from the wafer surface with heat.

An array of processing apparatus has been developed to carry out each of these steps. Mature technologies include coating and development tracks capable of a throughput of hundreds of wafers per hour and batch tanks with the capacity to clean up to 100 wafers at a time. As the resolution of typical process nodes and the prevalence of high-aspect ratio (HAR) features has increased, there have been corresponding increases in the fragility of surface patterns and the strictness of quality control required to produce functioning devices. These conditions have spurred the development and increasing adoption of single-wafer processing apparatus, including spin-coating tools, cleaners, baking modules, and drying stations.

### SUMMARY

A method of processing a substrate includes loading and processing the substrate in a process chamber, and obtaining first gas sensor data generated by interaction of a first target gas with a first gas sensor fluidly coupled to a headspace of the process chamber. The method includes determining a first metric for the processing based on the first gas sensor data, the determining including comparing the first gas sensor data with a first gas calibration data set. The method includes terminating the processing of the substrate based on the first metric for the processing.

An apparatus includes a process chamber, a wafer support disposed in the process chamber, a headspace above the wafer support, and a gas sensor fluidly coupled to the headspace and configured to generate gas sensor data.

A method of processing substrates includes performing a cyclic measurement process to generate a first gas calibration data set from the substrates. One cycle of the cyclic measurement process including: placing one of the substrates in a process chamber, obtaining first gas sensor data generated by interaction of a first target gas with a first gas sensor fluidly coupled to a headspace of the process chamber, removing the substrate from the process chamber, and performing a quality control test on the substrate and based thereon adding the first gas sensor data to a first gas calibration data set.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates in cross section a single-wafer processing tool, in which a wafer substrate is disposed below a mobile dispensing arm within a process chamber equipped with one or more gas sensors in any of several locations, according to various embodiments;

FIG. 2 illustrates in cross section a single-wafer drying tool, in which a wafer substrate rests on a support within a process chamber that may be equipped with a gas inlet and one or more gas sensors in any of several locations, according to various embodiments;

FIG. 3 illustrates in cross section a lidded batch tank for processing multiple substrates by immersion in a bath, the bath being disposed within a process chamber equipped with one or more gas sensors in any of several locations, according to various embodiments;

FIG. 4 illustrates in cross section a lidded batch tank for processing multiple substrates by immersion in a bath through which a process gas is additionally bubbled, the bath being disposed within a process chamber equipped with one or more gas sensors in any of several locations, according to various embodiments;

FIG. 5 illustrates in cross section a process apparatus appropriate for measuring outgassing, in which a wafer rests on a support within a process chamber equipped with a gas sensor, according to an embodiment;

FIG. 6 presents a block diagram of a wafer-processing apparatus in which any of the types of process apparatus illustrated in FIGS. 1-5 may be coupled to a controller and a memory containing a gas calibration data set and instructions for producing a metric on the process and process-control signals, according to various embodiments;

FIGS. 7A-7F illustrate different ways in which a comparison may be made between a measured concentration of target gas and calibration data, including comparisons with a single stored calibration value of the concentration (FIGS. 7A and 7B); comparisons with a stored calibration curve for the concentration at the same point in time (FIGS. 7C and 7D); and comparison by way of a time-correlation function (FIGS. 7E and 7F), according to various embodiments;

FIG. 8 provides a flow chart for a method of processing a substrate, in which gas sensor data may be compared with a gas calibration data set to determine a metric on the process, based whereon the process may be halted, according to an embodiment;

FIG. 9 provides a flow chart for a method of processing a substrate after applying the method of FIG. 8, in which second gas sensor data may be compared with a second gas calibration data set to determine a second metric on the process, based whereon the process may be halted, according to an embodiment;

FIG. 10 provides a flow chart for a method of processing substrates, in which a cyclic measurement process involving processing, measurement of gas sensor data, and quality control testing of the processed substrates may be used to assemble a gas calibration data set, according to various embodiments;

FIG. 11 provides a flow chart for a method of processing a substrate after applying the method of FIG. 10, in which second gas sensor data may be compared with a gas calibration data set to determine a first metric on the process, based whereon the process may be halted, according to an embodiment.

### DETAILED DESCRIPTION OF ILLUSTRATIVE EMBODIMENTS

The development of ever higher-resolution process nodes for semiconductor fabrication creates technical challenges at every stage and in every facet of production, from the environmental controls and tooling in the fab to the details of individual process steps and quality control tests on the processed wafers. Maintaining continuous throughput of large numbers of wafers with as many functioning devices (dies) as possible is crucial for the profitability of the industry.

As such, the suitability of any individual process step is ultimately determined by its effect on the throughput of processed wafers and on the fraction of wafers (and individual dies) that meet standards for quality control. A process that improves or maintains these statistical measures may be used at scale, while a process that degrades them will be modified or rejected.

With process nodes having reached the nanoscale—far smaller than the particulate contaminants that may be present in typical lab reagents—quality control standards require that the liquids and gases used in each process step be ultrapure and ultralow in particulates. Deionized (DI) water, for example, may have an electrical resistivity at or above 18.18 MΩ·cm at 25° C., close to the theoretical maximum for pure water and corresponding to a concentration of dissolved solutes on the order of parts per billion.

For example, in a single photoresist development step for a 300 mm wafer, as much as 100 mL of a stringently produced and purified developer may be used. Flowing 200 mL of ultrapure DI (UDI) water across the wafer surface may then be required before it rinses clean of excess developer and any developed portions of the surface pattern (in the sense of a final sampled portion of the rinsate registering near-maximal resistivity). Because each wafer may serve as substrate for dozens of component layers, each of which may in turn be produced through a lengthy series of steps entailing the use (and subsequent removal) of a variety of process chemicals, the material requirements are considerable. Thousands of gallons of ultrapure DI water may be used to produce a single finished wafer, with millions of gallons consumed per day at a typical semiconductor fab. The cost of producing that water—not to mention the cost of producing (or purchasing and transporting) the other process chemicals—can only be compensated in part by recirculation, reclamation, and reuse.

Even so, many current surface preparation, processing, and cleaning systems—as well as the process steps they implement and the specific types and amounts of process chemicals they consume—have been developed without direct or indirect real-time process monitoring. As such, material consumption could be higher than required to avoid process risk. If, for example, monitoring could show that rinsing a wafer with only 100 mL of UDI water after a given processing step is sufficient to meet quality standards, yet typical practice uses 200 mL, then 50% of the water use in that step would be undetected waste. All waste has corresponding material and energetic costs and ultimately contributes to the environmental externalities of semiconductor fabrication, which are magnified when the waste includes hazardous reagents. Moreover, the associated financial costs represent hidden manufacturing inefficiencies.

Embodiments of the inventions disclosed herein relate to the integration of sensor technologies into wafer-processing apparatus for semiconductor manufacturing, and in particular the integration of one or more gas sensors. In some embodiments, a gas sensor may be a micro-electromechanical systems (MEMS) device configured to detect a single gaseous species (or target gas). Advantageously, MEMS devices are small (e.g., thumbnail-sized), consume minimal power, have no moving parts, and allow for easy readout and calibration of the associated current, including by tandem integration of physically isolated reference circuit. In other embodiments, a gas sensor may be an electronic nose (e-nose) configured to detect and discriminate among several target gases simultaneously, according to the differential responses of a plurality of sub-sensors, which themselves may be MEMS devices. In still other embodiments, a mix of MEMS devices and e-noses may be integrated.

Specific MEMS devices appropriate for various embodiments of the present invention will be discussed in this description, but they are representative rather than exclusive examples and should not be construed to limit the type of MEMS device that may be selected for integration. In particular, the examples provided should not be understood to exclude any given mode of gas detection or any potential target gas. As such, the MEMS devices integrated may be electrochemical; chemiresistive, including devices based on electrolyte cells, polyelectrolyte membranes, or conjugated polymers, with or without metal dopants; optical, including photonic cavities or fiber-optic devices; acoustic, including surface acoustic wave (SAW) devices and mechanical resonators such as quartz crystal microbalances; or based on gas modulation of charge blockade in metal-oxide semiconductor (MOS) devices of p- or n-type, with or without doping. Other detection modes may also be appropriate, in various embodiments.

A wide variety of MEMS sensors devices and e-noses are commercially available and may be selected for embodiments of the present invention based on their reported specifications, performance, durability, etc. The only significant limitation on the choice of device given sensor is that its response time—the typical time required for the signal to rise to a specified threshold (usually 90% of total saturation) may be compatible with completion of at least one measurement over the timescale of typical semiconductor manufacturing process steps. In certain embodiments, such as for single-wafer processing, a process timescale may be between 30 seconds and 5 minutes; in other embodiments, such as for batch processing, a process timescale may instead be between 5 minutes and 2 hours. Shorter response times are advantageous, of course, as are short recovery times—the typical time required for the signal to drop to a specified threshold (usually 10% of total saturation) so that finer-resolution monitoring may be provided.

Irrespective of the particular number, kind, or mix of gas sensors chosen for use in a particular embodiment of the present invention, the monitoring capabilities integrated into the associated apparatus may have useful applications beyond endpoint detection for process steps (and the associated reduction in material waste). For example, sensor integration also enables fault monitoring (such as detecting process failure or equipment leaks, in some embodiments) as well as process learning and optimization (in other embodiments).

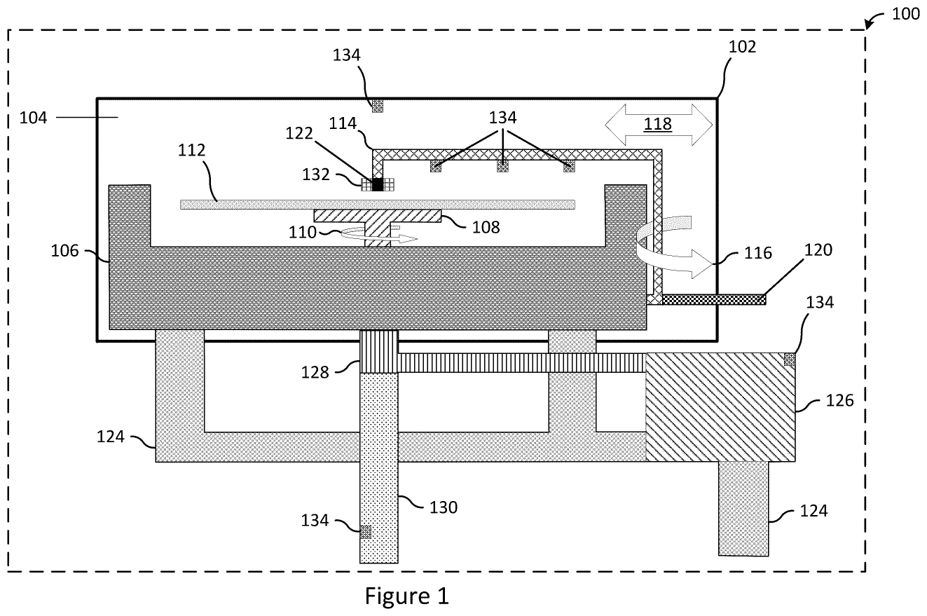

Several embodiments of the present invention will now be described with reference to FIG. 1, which provides a cross-sectional view of a single-wafer processing tool 100 appropriate for surface preparation, spray- or spin-coating, development, or cleaning. Tools of the type depicted, such as single wafer cleaning tools and wafer surface clean processing systems, have been adopted widely throughout the semiconductor industry over the last 25 years, because they are largely free of the equilibrium effects that reduce cleaning efficiencies in batch processes and because they require substantially smaller amounts of process chemicals to achieve comparable wafer outputs. Embodiments of the present invention may reduce those amounts further.

The single-wafer processing tool 100 comprises a process chamber 102 in which a volume of variable size allows for the unimpeded circulation and mixing of gas (or vapor), small droplets, aerosols, small particles, etc. This volume may be referred to as a headspace 104. In various embodiments, the single-wafer processing tool 100 may be constructed and configured for processing wafers of a variety of sizes, such as 300 mm wafers, 200 mm wafers, 50 mm wafers, and so on.

A basin 106 may be disposed within the process chamber 102 below a wafer support 108 upon which a wafer 112 rests. In some embodiments, the wafer support 108 may be a stationary platform. In other embodiments, the wafer support 108 may be a rotating chuck that may be configured to spin around its central axis (as indicated by a small curved arrow 110) at a constant or a variable rate (or to remain stationary). In these and other embodiments, the wafer support 108 may also incorporate a heating element for temperature control, a vacuum suction system for securing and stabilizing the wafer 112, electrodes for applying electrostatic fields, or other such mechanisms.

Wafer 112 refers generically to any suitable semiconductor workpiece being processed in accordance with embodiments of the present invention. The wafer 112 may be a bulk substrate such as a blank silicon wafer, a silicon-on-insulator (SOI) wafer, or any of various other semiconductor substrates. The wafer 112 may include any material portion or structure of a device, particularly a semiconductor or other electronics device, and is not limited to any specific base structure, to any defined number or type of photoresist or coating layers, or to any particular patterning (or lack thereof). Rather, the wafer 112 may include any such base substrate, layer, or patterning, and any combination thereof. The wafer 112 may also include chemicals from one or more processing steps, fragments of uniform or patterned layers that have previously been stripped or etched, and/or particulate contaminants.

A dispensing arm 114 may be disposed above the wafer 112. In some embodiments, the dispensing arm 114 may be configured along its length with a passthrough for one or more sets of tubing (not shown) to allow the separate or simultaneous flow of process chemicals, the composition of any such tubing being chosen according to its suitability for use with the process chemicals in question. In other embodiments, the dispensing arm 114 itself may be hollow in order to allow the flow of process chemicals, with an interior lining or overall composition chosen for durability on exposure to those process chemicals. Depending on the specifics of a particular application of the embodiments described here, process chemicals may be delivered to the dispensing arm 114 as neat substances or in the form of defined process solutions. Similarly, the process chemicals may be delivered in sequence or in parallel and in one or multiple steps, according to any desirable process protocol.

In some embodiments, the dispensing arm 114 may be equipped with a mechanism such that it may swing and describe an arc across the surface of the wafer 112, as indicated by a large curved arrow 116. In other embodiments, the dispensing arm 114 may move laterally across part or all of the diameter of the wafer 112, as indicated by the double-sided arrow 118. In still other embodiments, the combination of the arcing motion indicated by large curved arrow 116 and the lateral motion indicated by double-sided arrow 118 may allow rastering of the dispensing arm 114 across the wafer 112, such that any desired portion of the dispensing arm 114 may be disposed above any arbitrary point of the wafer 112.

The dispensing arm 114 (and any internal tubing, in accordance with an embodiment) may be connected to a material inlet 120 that is configured to deliver one or more process chemicals, which process chemicals may be neat substances or defined process solutions. In some embodiments, those process chemicals may be liquid; in other embodiments, they may be gaseous; in still other embodiments, a mix of process chemicals may be provided in either phase. (Whatever portions of the dispensing arm 114 may be configured to deliver gaseous process chemicals may be referred to collectively as a gas inlet.) These chemicals may be conducted through the dispensing arm 114 (and any internal tubing, in accordance with an embodiment) to a nozzle 122.

The nozzle 122 may dispense one or more process chemicals onto the wafer 112, doing so in sequence or in parallel and in one or multiple steps, according to any desirable process protocol. The nozzle 122 may be heated, pressurized, or otherwise configured for delivery of the process chemicals to proximate portions of the wafer 112 in the manner most suitable for a given process step.

During and subsequent to dispensing of any process chemicals from nozzle 122 onto the wafer 112, a variety of physical phenomena and/or chemical reactions may occur. In some embodiments, the process chemicals dispensed may be used to prepare a pristine (or previously coated or patterned) surface of the wafer 112 for further treatment. In other embodiments, the process chemicals dispensed from nozzle 122 may flow over the wafer 112 and drip into the basin 106, rinsing the wafer 112 of chemicals left over from one or more previous processing steps; fragments of uniform or patterned layers that have previously been stripped or etched; and/or particulate contaminants. In these and other embodiments, the process chemicals may also react with the surface of the wafer 112, with the effect of displacing, degrading, or combining with previous coatings or patternings or the aforementioned leftover chemicals, layer fragments, and/or particulates. In some embodiments, these reactions may generate additional process residues. Some portion of the process chemicals, their residues, leftover chemicals from previous processes, layer fragments, and/or particulates present in the system may be in the liquid phase, eventually flowing into the basin 106, while other portions may be gaseous or may evaporate from liquid to enter the headspace 104.

Similarly, in some embodiments, the centrifugal force imparted to dispensed liquids by the wafer support 108 as it spins around its central axis (as indicated by small curved arrow 110) may cause them to form a thin layer or coating on the wafer 112 that dries by evaporation of a solvent, the resulting solvent vapor entering the headspace 104. In other embodiments, process chemicals contacting the surface of the wafer 112 as heated by a temperature-controlled version of the wafer support 108 may evaporate, spatter, or otherwise produce droplets, aerosols, or gases that may be suspended in the headspace 104. Other droplets, aerosols, or gases may simply escape from the nozzle 122 and waft through the headspace 104 without ever having contacted the wafer 112.

Any liquid entering the basin 106 exits the process chamber 102 through a drain system 124, which may be open continuously or equipped with switchable plugs (not shown), according to an embodiment. The drain system 124 may work solely by gravity, or it may be equipped with one or more pumps in order to facilitate flow of liquid from the basin 106. The drain system 124 may also be connected inline to a catch tank 126, which may collect a sufficient volume of liquid waste to prevent backflow from the drain system 124 into the basin 106 or bursting of the drain system 124 itself (in the event that the flow rate of process chemicals into the basin 106 exceeds the maximum flow rate of the drain system 124). When less than completely full, the catch tank 126 may itself contain a volume of gas evaporated from the liquid waste (not shown). Any such volume may be coupled to the headspace 104 by through-connection via an exhaust system 128.

The exhaust system 128 may be coupled to the headspace 104 (and to any gas volume within the catch tank 126) by vents (not shown). The exhaust system 128 may further be equipped with switchable valves (not shown) allowing the exhaust system 128 to exert negative pressure relative to the headspace 104 of the process chamber 102, preventing gas buildup within the process chamber 102 and allowing for continuous airflow when suitable for a given process protocol. Gas transported through the exhaust system 128 eventually may reach and be vented through a gas outlet 130.

The headspace 104, any gas volume within the catch tank 126, and the interior, gas-conducting volume of the exhaust system 128 may be fluidly coupled to each other and to any gas sensors present. As an example, various gases present in these portions of the single-wafer processing tool 100 may mix, intermingle, or interact with each other and/or with adjacent components of the single-wafer processing tool 100 by bulk flow, diffusion, and/or other physical processes, under the influence of (or independent from) suction or negative pressure. Gas abundances in the headspace 104 may reflect most directly the process chemicals being applied to the wafer 112 and any products of chemical or physical processes occurring on the surface of the wafer 112, while gas abundances in any gas volume within the catch tank 126 may reflect most directly the composition of liquid waste collected within the catch tank 126 from the drain system 124. Gas abundances within the gas outlet 130 of the exhaust system 128 may correlate with the amounts of the process chemicals and other substances present throughout the single-wafer processing tool 100, excepting the internal spaces of the dispensing arm 114 and the material inlet 120.

Gas sensors, including MEMS devices and e-noses, may be integrated with the single-wafer processing tool 100 separately or in combination and at a multiplicity of locations within the process chamber 102, the catch tank 126, or the exhaust system 128, including the gas outlet 130, according to various embodiments. In some embodiments, one or more gas sensors may be incorporated into a sensor collar 132 that surrounds the nozzle 122, proximate to the point of process chemical delivery on the wafer 112. In other embodiments, one or more gas sensors may be placed elsewhere along the dispensing arm 114 (as indicated by gas sensors 134) or on a separate sensing arm (not shown). In embodiments incorporating a distinct sensing arm, that sensing arm may be fixed. In other such embodiments, the sensing arm may describe an arc through a swinging motion akin to that indicated by large curved arrow 116. In still other such embodiments, the sensing arm may move laterally across part or all of the diameter of the wafer 112 in a manner akin to that indicated by double-sided arrow 118. Some embodiments may allow the sensor arm to combine motions indicated by large curved arrow 116 and double-sided arrow 118 to raster across the wafer 112, such that any desired portion of the sensing arm may be disposed above any arbitrary point of the wafer 112.

Other embodiments may include individual gas sensors disposed along the inner wall of the process chamber, such as a gas sensor 134 placed directly above the center of the wafer 112 and the nozzle 122. In still other embodiments, a gas sensor 134 may be placed in the exhaust system 128 (including the gas outlet 130) or in the catch tank 126 of the drain system 124. Embodiments may also include a gas sensor (not shown) within whatever portion or portions of the material inlet 120 is dedicated to process gases.

In any of the embodiments described in which multiple gas sensors may be integrated with the single-wafer processing tool 100, the gas sensors may all be of the same type, or may include a mix of different functionalities, whether MEMS devices, e-noses, or any other suitable choice of gas sensor.

Embodiments of the present invention as described for the single-wafer processing tool 100 allow for numerous advantageous applications.

An embodiment includes determining an endpoint of a rinsing process by obtaining gas sensor data for process chemicals dispensed as part of a previous processing step. For example, embodiments may provide endpoint detection when rinsing isopropyl alcohol (IPA) from a wafer surface with ultrapure DI (UDI) water or rinsing dimethyl sulfoxide (DMSO) from a wafer surface with IPA or an IPA/UDI water mixture. Gas sensor data may be used to determine when the IPA or DMSO has been fully removed from the surface and thus to terminate the rinsing process. In the former case, the gas sensor(s) used may be any suitable VOC sensor; in the latter case, where distinguishing DMSO from IPA may be important, multiple MEMS gas sensors or an e-nose may be used.

Some embodiments may provide endpoint detection based on monitoring of process chemicals dispensed during the monitored process step. For example, embodiments incorporating a humidity sensor may be used to determine an endpoint for rinsing with UDI water when humidity in the process chamber 102 has saturated. Such embodiments may also be used to monitor processes in which UDI water is used to rinse a wafer bearing HAR features, and the water is subsequently displaced by IPA in order to stabilize the surface. In particular, these embodiments may provide real-time monitoring of the humidity and thus enable a precise determination of the point at which the rinsing liquid should be switched from UDI water to IPA.

Other embodiments further incorporating a VOC sensor, whether a MEMS sensor or an e-nose, may provide endpoint detection when rinsing a wafer surface bearing DMSO with an IPA/water mixture. Use of a VOC sensor to detect the IPA or a humidity sensor to detect the water may provide an indication as to when the process chamber 102 has saturated. Similarly, hot IPA drying processes could be monitored in order to trigger a fault when the amount of humidity or of IPA in the chamber rises or falls too quickly relative to a gas calibration data set. If the IPA concentration falls off too quickly, for example, there may be unexpected condensation in the material inlet 120 or the dispensing arm 114 before the hot IPA vapor reaches the wafer 112.

Still other embodiments incorporating sensors configured to detect other gases may also be used to provide fault detection (indicating a problem with the monitored process, rather than its end). When cleaning with the SC1 step of the RCA clean, for example, the ratio of ammonium hydroxide (NH₄OH) to hydrogen peroxide (H₂O₂) must not become too high, or the ammonium hydroxide may etch the wafer surface. Loss of hydrogen peroxide can also correlate with decay of hydrogen peroxide to form oxygen gas, which can form bubbles at high enough concentration, providing a mechanical getter for particulates that can redeposit on the wafer. If these bubbles escape from the solution, the associated buildup of oxygen also presents a risk of fire or explosion. Ammonia can also evolve from the cleaning solution, especially at elevated temperature conditions. Consequently, embodiments incorporating gas sensors configured to detect ammonia and/or oxygen provide a means of maintaining target ratios of NH₄OH and H₂O₂ in the SC1 solution, thereby reducing the likelihood of unintended etching, while also allowing for a fault to be triggered if conditions otherwise depart from standards.

Additional embodiments incorporating gas sensors configured to monitor chemicals dispensed during the monitored process step enable process learning and optimization. Advanced cleaning and stripping methods incorporate ozonated liquids, including UDI water and sulfuric acid (H₂SO₄) solutions. In conventional systems, ozone (O₃) concentrations at the surface of the wafer 112 are unknown, and losses to process efficiency from degassing of O₃ are not characterized. Moreover, the extent to which O₃ may degrade to oxygen (O₂) and present a risk of combustion are not well understood. In various embodiments, gas sensors are used to detect typical ozone concentrations near the surface of the wafer and to optimize the process recipe to minimize degassing. For these purposes, gas sensors configured to detect ozone may be used.

In further embodiments, byproducts of the current process step (rather than process chemicals dispensed) are monitored. For example, when stripping organic materials such as photoresists with piranha solutions, in which sulfuric acid (H₂SO₄) and hydrogen peroxide (H₂O₂) are combined to form Caro's acid (H₂SO₅), the oxidation of the organic material produces carbon dioxide (CO₂). The process endpoint may be determined by observing the cessation of CO₂ production in gas sensor data. For this purpose, a gas sensor configured to detect CO₂ may be used.

Etching silicon nitride with phosphoric acid (H₃PO₄) produces ammonium ions (NH₄+) that may evolve from the surface as gaseous ammonia (NH₃). The process endpoint may be determined by observing the cessation of NH₃ production in gas sensor data. For this application, a gas sensor configured to detect ammonia may be used. Similarly, etching polysilicon or silicon nitride with tetramethylammonium hydroxide (TMAH) produces hydrogen gas (H₂) that may evolve from the surface. The process endpoint may be determined by observing the cessation of H₂ production in gas sensor data using a gas sensor configured to detect hydrogen. In the latter case, the monitoring also has implications for process safety, because of the flammability of H₂ gas and the potential for explosion if too much gas builds up in the single-wafer processing tool 100.

Another example of byproduct monitoring comes from the etching or stripping of silicon dioxide (SiO₂) films with dilute hydrofluoric acid (DHF), which produces water that may evolve from the surface. The process endpoint may be determined by observing humidity returning to ambient levels using a humidity sensor.

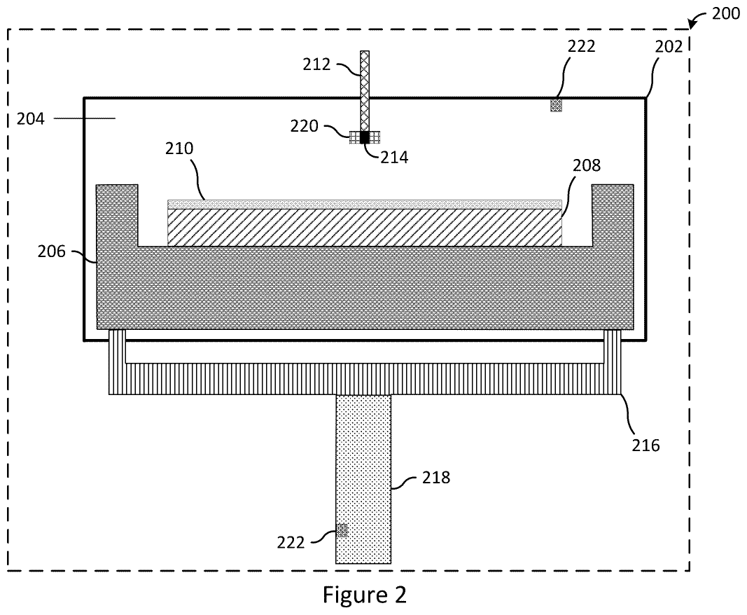

Embodiments described thus far have involved integration of sensor technologies with single-wafer processing tools of the type described in FIG. 1, but the present invention is by no means limited in scope to such tooling. Other embodiments may integrate sensors into a single-wafer drying station. Several such embodiments will now be described with reference to FIG. 2, which provides a cross-sectional view of a single-wafer drying station 200, which may correspond (in certain embodiments) to a conventional hot plate or (in others) to a sophisticated supercritical dryer.

Supercritical dryers have been increasingly widely adopted in the semiconductor industry as a response to new fabrication challenges. Even as process nodes have achieved higher resolutions, reducing the critical dimension (CD) of features patterned on the wafer surface, devices have become more complex. For example, in certain memory devices, such as DRAM and 3D NAND flash, contacts may be formed across many layers of patterning. The corresponding increase in the height of a via relative to its width CD makes such high-aspect ratio (HAR) structures susceptible to the phenomenon of pattern collapse, in which adjacent lines buckle against each other and may fuse irrecoverably. Pattern collapse is an acute risk during drying, when displacement or evaporation of high-surface tension solvents (such as UDI water or IPA) exerts unbalanced forces on HAR features.

Supercritical dryers work by flowing CO₂ over the wafer surface at temperatures and pressures above the critical point (30.98° C. and 72.79 atm), such that there is no physical distinction between liquid and gas and thus no surface tension exerted by this warm, dense working fluid. As the supercritical CO₂ circulates, solvents such as IPA are gently removed from the wafer surface through formation of a supercritical mixture. Once the supercritical mixture has formed, it may be vented, with additional CO₂ used to flush the system. The embodiments now to be described provide confirmation of supercritical mixing and solvent removal, namely, confirmation of completed drying, allowing termination of the drying as soon as practicable and an increase in throughput of the drying station.

The single-wafer drying station 200 comprises a process chamber 202 in which a volume of variable size allows for the unimpeded circulation and mixing of gas (or vapor), small droplets, aerosols, small particles, etc. This volume may be referred to as a headspace 204.

A basin 206 may be disposed within the process chamber 202 below a wafer support 208 upon which a wafer 210 rests. In some embodiments, the wafer support 208 may be a simple platform; in other embodiments, the wafer support 208 may be a hot plate or other temperature-controlled device for heating the wafer. In these and other embodiments, the wafer support 208 may also incorporate a vacuum suction system for securing and stabilizing the wafer 210 or other such mechanisms. Wafer 210 may be a wafer in the same sense as wafer 112.

The process chamber 202 may be equipped with a gas inlet 212, which may be disposed above the wafer 210 (in some embodiments) or elsewhere (in others). In some embodiments, gas inlet 212 may be configured to deliver CO₂ through the nozzle 214 into the process chamber 202 and thus into the headspace 204, with continued flow of gas leading to increased pressures within the process chamber 202. In certain embodiments, the CO₂ may be delivered to the process chamber 202 at a temperature between 20° C. and 100° C., such that the CO₂ may eventually reach supercriticality. In other embodiments, the gas inlet 212 and the nozzle 214 may be absent, or the gas inlet 212 may be configured to deliver non-reactive gas (such as N₂ or Ar). In embodiments including the gas inlet 212 and the nozzle 214, the latter may be heated, pressurized, or otherwise configured in the manner suitable for the drying protocol.

During and subsequent to dispensing of any gas from nozzle 214 into process chamber 202, a variety of physical phenomena may occur, including the formation of a supercritical fluid of CO₂ or a supercritical mixture of CO₂ and any solvents present on the wafer 210, such as UDI water or IPA. Before the formation of any supercritical mixture, any liquids present in the process chamber 202, such as solvents present on the surface of the wafer 210, may evaporate to enter headspace 204.

An exhaust system 216 may be coupled to the headspace 204 by vents (not shown). The exhaust system 216 may further be equipped with switchable valves (not shown) allowing the exhaust system 216 to exert negative pressure relative to the headspace 204 of the process chamber 202. Gas transported through the exhaust system 216 eventually may reach and be vented through a gas outlet 218.

The headspace 204 and the interior, gas-conducting volume of the exhaust system 218 may be fluidly coupled to each other and to any gas sensors present. Gas abundances in the headspace 204 may reflect most directly the process gases being delivered to the process chamber 202 and any products of chemical or physical processes occurring on the surface of the wafer 210, while gas abundances within the gas outlet 218 of the exhaust system 216 may correlate with the amounts of the process gases and other substances present throughout the single-wafer drying station 200, excepting the internal spaces of the gas inlet 212. In embodiments corresponding to a supercritical dryer, a transition from detectable to undetectable solvent in the gas outlet 218 may indicate the formation of a supercritical mixture.

Gas sensors, including MEMS devices and e-noses, may be integrated with the single-wafer drying station 200 separately or in combination and at a multiplicity of locations within the process chamber 202 or the exhaust system 216, including the gas outlet 218, according to various embodiments. In some embodiments, one or more gas sensors may be incorporated into a sensor collar 220 that surrounds the nozzle 214. Other embodiments may include individual gas sensors disposed along the inner wall of the process chamber, such as a gas sensor 222 placed above the edge of the wafer 210. In still other embodiments, a gas sensor 222 may be placed in the exhaust system 216 (including the gas outlet 218), as illustrated. Embodiments may also include a gas sensor (not shown) within the gas inlet 212.

In any of the embodiments described in which multiple gas sensors may be integrated with the single-wafer drying station 200, the gas sensors may all be of the same type, or may include a mix of different functionalities, whether MEMS devices, e-noses, or any other suitable choice of gas sensor.

Embodiments of the present invention as described for the single-wafer drying station 200 enable advantageous applications, in particular for monitoring the onset of supercriticality. Real-time monitoring of the phase transition through the proxy measurement in the process chamber 202 may contribute to process optimization and thereby reduce cycle time and defects in the processed wafers. Embodiments including one or more VOC sensors whether MEMS sensors or e-noses, may be used to detect solvents such as IPA. Other embodiments may include sensors configured to monitor CO₂ in the system and to trigger a fault in the event of an unexpected loss of pressure. Still other embodiments may monitor both the solvent and the CO₂. While durability testing may be required to determine whether a given sensor may withstand the elevated pressure conditions in the process chamber 202, gas sensors may be placed freely in the exhaust system 216 (including the gas outlet 218).

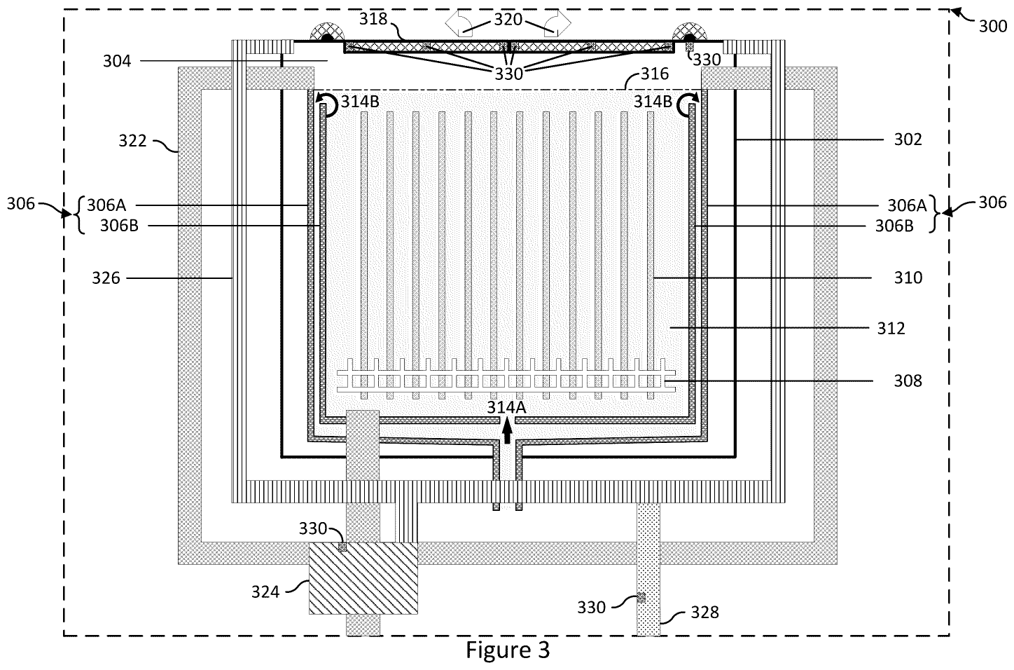

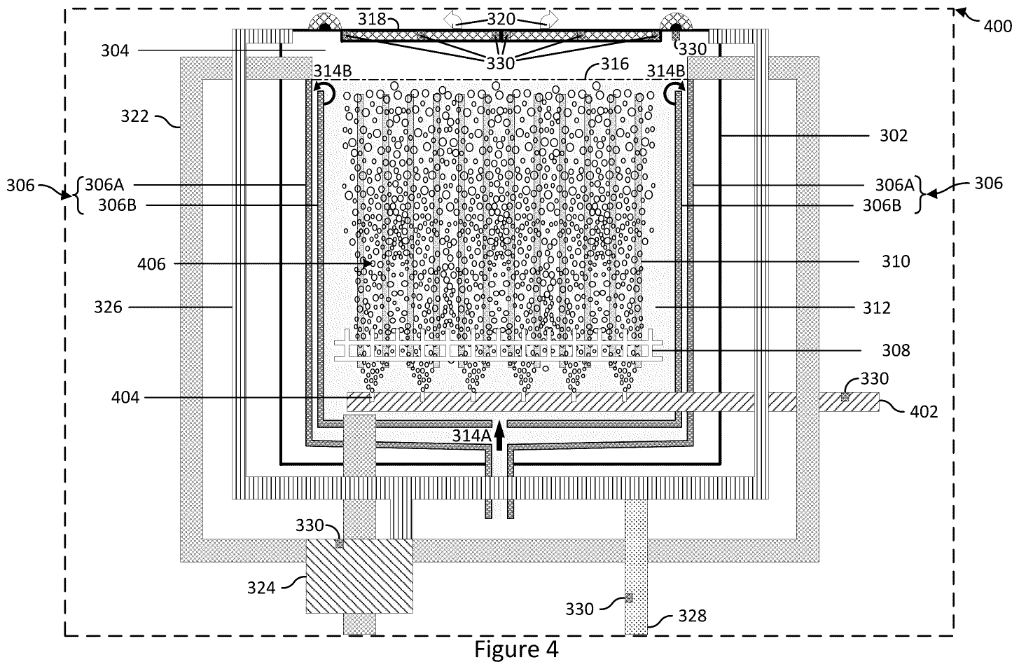

Notwithstanding the focus so far in this description on single-wafer processing, batch processing of wafers continues to be widely used in batch cleaning systems and automatic wet stations, especially for cleaning steps such as the “simple clean 1” (SC1) of the RCA clean process. Batch processing may also be indicated for modern processes such as Marangoni (hot IPA) drying. We now describe the operation of a batch processing tool 300 (respectively, 400) with reference to FIGS. 3 and 4. The principal difference between these figures is the inclusion in FIG. 4 of a gas inlet 402 equipped with one or more gas outlets or bubblers 404 that may produce a multiplicity of bubbles of one or more process gases within a bath solution 406, as appropriate for a given process protocol, in accordance with an embodiment. That being the case, identical reference numerals will be used for like components in each figure.

The batch processing tool 300 (400) comprises a process chamber 302 in which a volume of variable size allows for the unimpeded circulation and mixing of gas (or vapor), small droplets, aerosols, small particles, etc. This volume may be referred to as a headspace 304.

A batch tank 306 may be disposed within the process chamber 302, with sub-components 306A and B corresponding to its outer and inner walls. In some embodiments, the batch tank 306 may be a recirculating tank with channels disposed between the outer walls 306A and the inner walls 306B, as illustrated in FIGS. 3 and 4. In other embodiments, the batch tank 306 may be solid except at the material inlet 314A, allowing the introduction of process chemicals from the bottom of the tank, as indicated by the large black arrow. (In some embodiments, there may be several such inlets, though only one is shown in FIGS. 3 and 4.) Within the batch tank 306, a wafer support or rack 308 may be disposed and configured to hold a plurality of wafers 310. While FIGS. 3 and 4 depict 12 wafers loaded into the rack, this number was chosen as a matter of illustration and should not be understood as limiting. In typical embodiments, and depending on the physical dimensions of the individual wafers to be processed and the details of a given processing step, the number of wafers that may be accommodated in the batch tank 306 at one time may be as few as 3 and as many as 100. Wafers 310 may be wafers in the same sense as wafer 112.

The batch tank 306 may be filled from below with process chemicals entering via the material inlet 314A. In some embodiments, the material inlet may be disposed elsewhere than at the bottom of the batch tank 306. Process chemicals may be liquid (as in SC1 processing) or gaseous (as in the Marangoni process), according to various embodiments.

In some embodiments, the process chemicals dispensed through material inlet 314A may flow over the plurality of wafers 310 and lap over the edges of the inner walls 306B, entering the hollow cavity separating the inner walls 306B from the outer walls 306A as indicated by curved black arrows 314B and falling to the bottom of the hollow cavity, toward the material inlet 314A, to be recirculated. In embodiments in which the batch tank 306 is solid and one or more process chemicals is liquid, processing protocols may ensure that filling of the tank ceases when the tank is full, as indicated by the dot-dashed line at a surface 316 of the bath solution 312.

During and subsequent to filling of the tank through material inlet 314A, a variety of physical phenomena and/or chemical reactions may occur. In some embodiments, the process chemicals dispensed may be used to prepare pristine (or previously coated or patterned) surfaces of the plurality of wafers 310 for further treatment. In other embodiments, process chemicals may react with the surfaces of the plurality of wafers 310, with the effect of displacing, degrading, or combining with the previous coatings or patternings or the aforementioned leftover chemicals, layer fragments, and/or particulates. In some embodiments, these reactions may generate additional process residues. Some portion of the process chemicals, their residues, leftover chemicals from previous processes, layer fragments, and/or fragments present in the system may be in the liquid phase, in some embodiments recirculating through the hollow cavity in the batch tank 306, while other portions may be gaseous or may evaporate from liquid to enter the headspace 304.

In order to facilitate loading of the batch tank 306, and in order to protect against splashing or spattering from the surface 316, the batch processing tool 300 (400) may be equipped with a lid 318. In embodiments in which the process chemicals are gaseous or the gas inlet 402 may be included, the lid 318 also protects against loss of process gas from the headspace 304 of the process chamber 302. In FIGS. 3 and 4, the lid 318 is depicted as a hinged double lid opening outward from the top center of the batch processing tool 300 (400), as indicated by the angled arrows 320, in accordance with some embodiments. Other embodiments may equip the batch processing tool 300 (400) with a different lid construction and/or placement.

In order to protect against spillage of liquid process chemicals out of the batch tank 306 and into other parts of the process chamber 302 (402), and to allow for termination of the batch process, cleaning of the batch tank, etc., the batch processing tool 300 (400) may be equipped with a drain system 322, which may be open continuously or equipped with switchable plugs (not shown), according to an embodiment. The drain system 322 may be connected to the bottom of the batch tank 306 to allow for draining of the entire tank. The drain system 322 may also have drains disposed proximate to the top of the outer walls 306A, such that any overfill of process chemicals that cannot be caught and recirculated may be drained. The drain system 322 may work solely by gravity, or it may be equipped with one or more pumps in order to facilitate flow of liquid from the process chamber 302 and especially from the batch tank 306. The drain system 322 may also be connected inline to a catch tank 324, which may collect a sufficient volume of liquid waste to prevent backflow from the drain system 322 into the batch tank 306 or the process chamber 302. The presence of the catch tank 324 may also prevent bursting of the drain system 322 in the event that the flow rate of process chemicals into the batch tank 306 exceeds the maximum flow rate of the drain system 322. When less than completely full, the catch tank 324 may itself contain a volume of gas evaporated from the liquid waste (not shown). Any such volume may be coupled to the headspace 304 by through-connection via an exhaust system 326.

The exhaust system 326 may be coupled to the headspace 304 (and to any gas volume within the catch tank 324) by vents (not shown). The exhaust system 326 may further be equipped with switchable valves (not shown) allowing the exhaust system 326 to exert negative pressure relative to the headspace 304 of the process chamber 302, preventing gas buildup within the process chamber 302 and allowing for continuous airflow when suitable for a given process protocol. Gas transported through the exhaust system 326 eventually may reach and be vented through a gas outlet 328.

The headspace 304, any gas volume within the catch tank 324, and the interior, gas-conducting volume of the exhaust system 326 may be fluidly coupled to each other and to any gas sensors present. (In embodiments in which the process chemicals introduced through the material inlet 314A are gaseous, the interior volume of the batch tank 306 is also fluidly coupled to the aforementioned other volumes.) Gas abundances in the headspace 304 may reflect most directly the process chemicals bathing the plurality of wafers 310 and any products of chemical or physical processes occurring on the surfaces of the wafers 310, while gas abundances in any gas volume within the catch tank 324 may reflect most directly the composition of the liquid waste collected within the catch tank 324 from the drain system 322. Gas abundances within the gas outlet 328 of the exhaust system 326 may correlate with the amounts of the process chemicals and other substances present throughout the batch processing tool 300 (400), excepting the internal spaces of the material inlet 314A and (if present) the gas inlet 402.

Gas sensors, including MEMS devices and e-noses, may be integrated with the batch processing tool 300 (400) separately or in combination and at a multiplicity of locations within the process chamber 302, the catch tank 324, or the exhaust system 326, including the gas outlet 328, according to various embodiments. In some embodiments, one or more gas sensors 330 may be incorporated into the lid 318, proximate to the surface 316 of the bath solution 312. (In embodiments with a different lid construction or placement than that shown in FIGS. 3 and 4, the disposition of gas sensors relative to the surface 316 of the bath solution 312 may vary.)

Other embodiments may include individual gas sensors disposed along the inner wall of the process chamber, such as a gas sensor 330 placed above the edge of the batch tank 306. In still other embodiments, the gas sensor 330 may be placed in the exhaust system 326 (including the gas outlet 328), or in the catch tank 324 of the drain system 322, as illustrated. Embodiments may even include a gas sensor 330 within the gas inlet 402, if present.

In any of the embodiments described in which multiple gas sensors may be integrated with the batch processing tool 300 (400), the gas sensors may all be of the same type, or may include a mix of different functionalities, whether MEMS devices, e-noses, or any other suitable choice of gas sensor.

Embodiments incorporating a VOC sensor, whether a MEMS sensor or an e-nose, may be appropriate for monitoring hot IPA drying processes, with a fault being triggered when the amount of humidity or of IPA in the process chamber 302 rises or falls too quickly relative to a gas calibration data set. If the IPA concentration falls off too quickly, for example, there may be unexpected condensation in the material inlet 314A before the hot IPA vapor reaches the plurality of wafers 310. Embodiments incorporating sensors configured to detect ammonia and/or oxygen may be used for the SC1 step of the RCA clean, thereby preventing unwanted etching or dangerous conditions from oxygen buildup in the chamber.

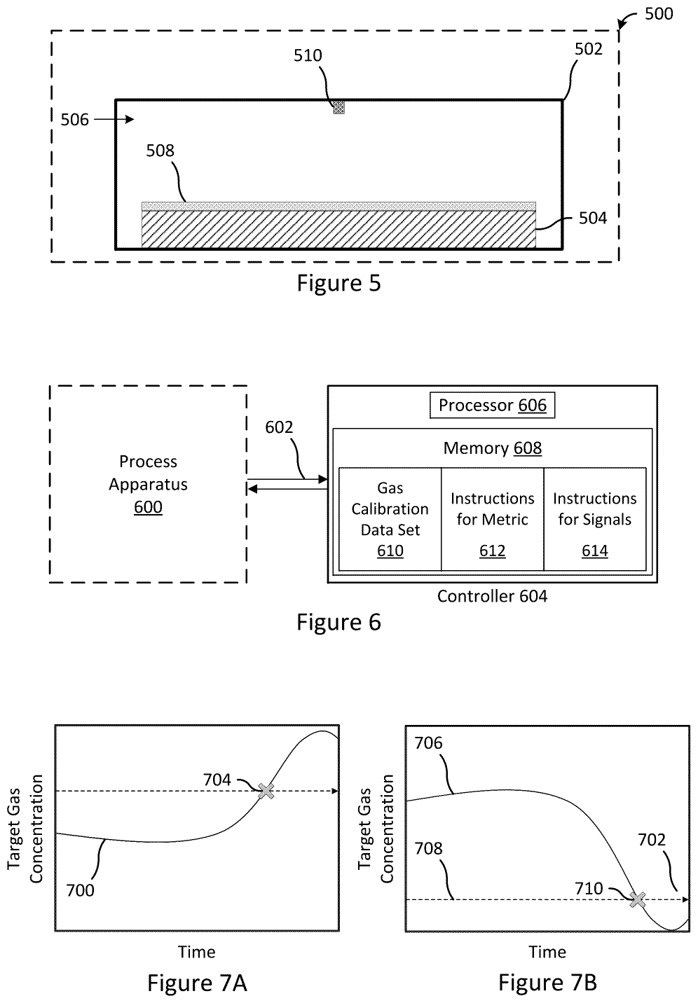

FIG. 5 illustrates a process apparatus 500, in accordance with an embodiment. In process apparatus 500, a process chamber 502 is equipped with a wafer support 504 upon which a wafer 508 rests. (The wafer 508 may be a wafer in the same sense as wafer 112.) A volume of variable size above the wafer support 504 allows for the unimpeded circulation and mixing of gas (or vapor), small droplets, aerosols, small particles, etc. This volume may be referred to as a headspace 506. The headspace 506 may be fluidly coupled to a gas sensor 510 (such as a MEMs device or an e-nose) disposed along the inner wall of the process chamber 502. Gas abundances in the headspace 506 may reflect the gases charged within the process chamber at time of loading, if any, according to an embodiment. In other embodiments, gas abundances in the headspace may reflect chemical or physical processes occurring on the surface of the wafer 508, such as outgassing. Indeed, a process apparatus of the type depicted in FIG. 5 might be used to characterize the nature and time evolution of outgassing in a wafer 508 at various stages of fabrication.

A controller coupled to the gas sensor may be programmed to receive signals from any gas sensors present, interpret those signals as pressure or concentration data, convert those data into metrics for the underlying processes, and generate feedback signals (when appropriate) triggering the modification or termination of said processes. Such a controller is now described with reference to FIG. 6.

In FIG. 6, a process apparatus 600 is electrically coupled (as depicted by the double arrow) 602 to a controller 604. (The process apparatus may be the single-wafer processing tool 100, the single-wafer drying station 200, the batch processing tool 300 (400), the process apparatus 500, or indeed other tooling, according to various embodiments.) The controller may include a processor 606 and a memory 608. The memory 608 may store a gas calibration data set 610 and instructions 612 for generating a metric on the underlying process from any signals measured through the electrical coupling 602 (according to a comparison with the gas calibration data set 610). The memory 608 may also store instructions 614 for generating and transmitting control signals to the process apparatus 600. Control signals may include those used to start or stop a given process, as well as those used in the course of running said process, such as by opening or closing valves or by modifying rates for material flow; by opening or closing plugs or vents connecting a process chamber to a drain or exhaust system; by changing the pressure exerted relative to the process chamber through an exhaust; by changing the temperature, rate of rotation, and/or suction of a wafer support such as a rotating chuck or hot plate; and so on, according to various embodiments.

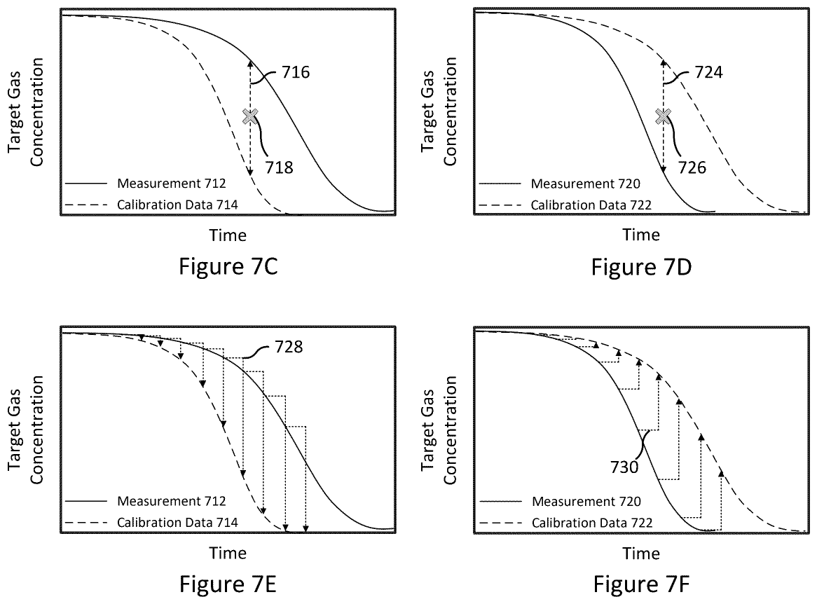

Comparisons between gas sensor data and the gas calibration data set 610 used to generate the metric according to instructions 612 may be any of several different kinds, or a combination thereof, or any other suitable comparison, according to various embodiments. Several such types of comparison are provided in FIGS. 7A-7F. While the y-axis labels in FIGS. 7A-7F refer to target gas concentrations, in some embodiments the data to be compared may be gas pressures or other quantities suitable for a given process step. Accordingly, the labeling of these Figures should not be understood as limiting.

A comparison between the most recently measured gas data and a stored threshold value may be used in some embodiments. With reference to FIG. 7A, the gas data 700 may be compared to a stored value 702, with an event 704 being triggered whenever the gas data 700 first exceed the stored value 702. (In other embodiments, the event 704 may be triggered if the gas data 700 exceed the stored value 702 for a longer time than a stored, specified duration.) Applications for which embodiments of this type may be useful are detecting loss of ammonium hydroxide or hydrogen peroxide generating excess ammonia, water, or oxygen in the SC1 clean solution, detecting degradation of ozone to generate excess oxygen in the clean, and so on. With reference to FIG. 7B, the gas data 706 may be compared to a stored value 708, with an event 710 being triggered whenever the gas data 706 first fall below the stored value 708. (In other embodiments, the event 710 may be triggered if the gas data 706 fall below the stored value 708 for a longer time than a stored, specified duration.) Applications for which embodiments of this type may be useful are detecting loss of IPA in the hot IPA drying process or detecting the transition to supercriticality in the supercritical dryer. Any embodiment in which a gas sensor is placed in a gas inlet of the apparatus may also be used for fault detection in this way.

In other embodiments, it may be more useful to compare measured data with a known “gold standard” of calibration data, and for an event to be triggered if the two curves diverge. With reference to FIG. 7C, the gas data 712 may be compared with a gas calibration data set 714, with an event 718 being triggered whenever the difference between the two curves exceeds a stored value 716. In FIG. 7C, the gas data 712 correspond to a higher concentration than expected from the gas calibration data set 714. FIG. 7D illustrates the opposite scenario, in which gas data 720 correspond to a lower concentration than expected from a gas calibration data set 722, such that the difference again exceeds a stored value 724, triggering an event 726. The stored value 716 (724) may be signed or unsigned, according to an embodiment. In certain embodiments, the event 718 (726) may also be triggered if the difference between the two curves exceeds the stored value 716 (724) for a longer time than a stored, specified duration. In other embodiments, the event 718 (726) may be triggered if a running average of a stored, specified number of the most recent measurements from the gas data 712 (720) diverges too much from the average over corresponding points in the gas calibration data set 714 (722). Applications in which embodiments of this type may be useful include humidity detection in drying and rinsing with UDI water.

A more general approach to comparison may be the use of time correlation functions (TCFs), as illustrated in FIGS. 7E and 7F for the same measurements and calibration data illustrated (respectively) in FIGS. 7C and 7D. Values of gas data 712 may be compared with values of a gas calibration data set 714 separated by a user-specified time delay. Suppose in particular that the gas data 712 and the gas calibration data set 714 have respective values D(t) and C(t) at a particular time t, with t=0 corresponding to the first point of the respective curves. Further suppose that the user-specified time delay is Δt. The values from the gas calibration data set 714 to be used for comparison may then be C(t+Δt), as indicated by the quarter-dotted lines and arrows 728. If no individual data point C(t+Δt) exists in the gas calibration data set 714, any suitable curve-fitting method may be used to provide an estimate, according to various embodiments.

Because similar reasoning may be applied to gas data 720 and gas calibration data set 722, yielding the comparisons indicated by quarter-dotted lines and arrows 730 in FIG. 7F, references below are made to elements of FIG. 7E. The principal distinction between the figures is which data represent larger concentrations at a given time t.

In typical embodiments, the time delay Δt may be non-negative, because the gas data 712 are collected during wafer processing, while the gas calibration data set 714 is stored. (In other words, gas calibration data set 714 may contain one or more values corresponding to the future of the current processing step, with t>τ, where τ represents the current time.) Nevertheless, in some embodiments (not illustrated), the time delay Δt may be chosen to be negative, as long as t+Δt remains non-negative. (Negative times precede the beginning of processing.)

The value of the time correlation function at the current time τ is TCF(τ; Δt)= [symbol] D(τ)C(τ+Δt) [symbol] , where the semicolon indicates parametric dependence of the time correlation function on the choice of time delay Δt. The angle brackets indicate a time average over the respective points of the gas data 712 and the gas calibration data set 714 (or, in the latter case and in some embodiments, to estimated points obtained from curve fitting methods). In typical embodiments, the time average may be taken from the beginning of the measurement (t=0) until the current time (t=τ). Other embodiments may employ a time average taken over a time window δt of times in the past of the current processing step, such that τ−δt<t<τ for the gas data 712 and τ+Δt−δt<t<τ+Δt for the gas calibration data set 714.

In various embodiments, it may be preferable to use a modified time correlation function. One such embodiment may employ a normalized time correlation function defined by dividing the above expression for TCF(t) by TCF(0). Other embodiments may modify TCF(t) by subtracting TCF(0), and still other embodiments may divide and then subtract TCF(0), such that the comparison may be based on relative deviations of the time correlation function from its initial value.

New values may be added to the time correlation function as further measurements are taken (corresponding to new values for the gas data 712 and thus new points in D(t)). As long as τ+Δt is within the range of times that may be present in the gas calibration data set 714, the time correlation function may be evaluated consistently using points from gas calibration data set 714 or (in some embodiments) using estimates from curve fitting. In some embodiments, it may also be desirable to allow for extrapolation beyond the last time represented in the gas calibration data set 714, in which case a suitable extrapolation method may be chosen (including, for example, evaluation of a polynomial fit to C(t)).

According to various embodiments, an event may be triggered if TCF(τ) diverges too much from a stored value, or if it diverges too much from a stored value for a longer time than a stored, specified duration. In other embodiments, an event may be triggered if TCF(τ) diverges too much (or for a longer time than a stored, specified duration) from the calculated value of the autocorrelation function (ACF) for the gas calibration data set 714. The autocorrelation function is defined analogously to the TCF but involves only the values C(t), ACF(τ; Δt)= [symbol] (C(τ)C(τ+Δt) [symbol] . In accordance with various embodiments, ACF(τ; Δt) may be modified for consistency with a selected version of the time correlation function.

In certain embodiments, an event may be triggered if the gas calibration data set 714 contains no points for t>τ, indicating that the current process time will exceed the expected duration. In still other embodiments, an event may be triggered if extrapolation methods have been used to provide one or more points for comparison for greater than a stored, specified number of measurement times.

In some embodiments, multiple gas calibration data sets akin to gas calibration data set 610 and multiple sets of instructions for generating a metric on the underlying process akin to instructions 612 may be stored within the memory. These additional data sets and instructions may be tailored for various underlying processes, target gases, gas sensor architectures, differing choices of comparison between measured gas data and calibration data, etc., according to the respective embodiments.

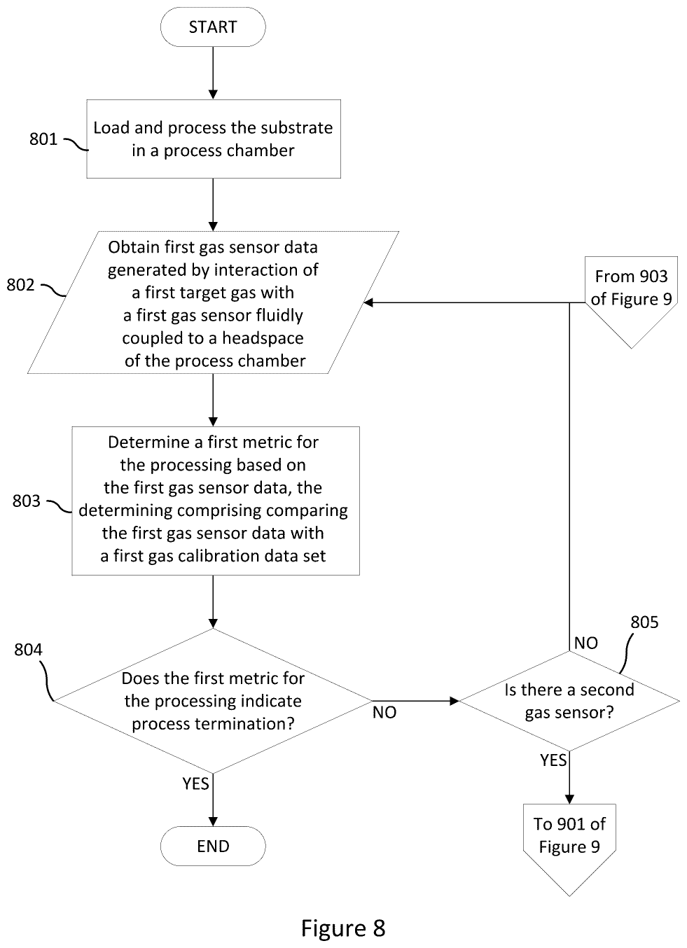

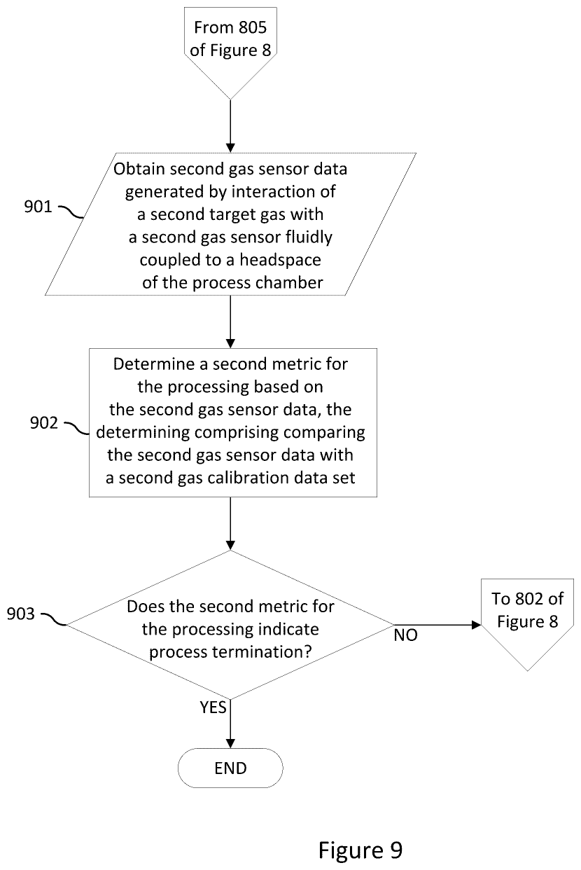

Given an embodiment of the present apparatus of the type indicated in FIG. 6, and incorporating a process apparatus of any of the types depicted in FIGS. 1-5 with a method for comparison such as those illustrated in FIG. 7, several methods for processing a substrate become possible. These methods are described below with reference to FIGS. 8-11.

FIG. 8 provides a flow chart for a method of processing a substrate, beginning by loading a substrate into a process chamber, such as any of the process chambers depicted in FIGS. 1-5 (see box 801 of FIG. 8). Processing of the substrate and other physical phenomena that may occur while the substrate is present in the chamber may produce a first target gas that interacts with a first gas sensor fluidly coupled to a headspace of the process chamber, which interaction results in first gas sensor data delivered to the controller 604 (see box 802 of FIG. 8). The controller 604 compares the first gas sensor data with a first gas calibration data set (corresponding to gas calibration data set 610) according to instructions for determining a metric on the processing (corresponding to instructions 612) in order to produce a first metric (see box 803 of FIG. 8). (The chosen method of comparison may be any of the comparisons illustrated in FIGS. 7A-7F, or it may be another method suitable for the chosen metric, according to various embodiments.) The controller 604 may then halt the processing based on the first metric, according to instructions for generating any necessary control signals (corresponding to instructions 614). In some embodiments, the controller may make a binary choice either to terminate processing of the substrate or to continue processing while monitoring the first target gas, corresponding to a simple loop over boxes 802-804 of FIG. 8. In other embodiments, and as illustrated in FIG. 8, a second gas sensor may be present in the process chamber, and the decision to terminate the processing may be conditioned on a subsequent method illustrated in FIG. 9.

FIG. 9 provides a flow chart for a method of processing a substrate after applying the method of FIG. 8. Processing of the substrate and other physical phenomena that may occur while the substrate is present in the chamber may produce a second target gas that interacts with a second gas sensor fluidly coupled to a headspace of the process chamber, which interaction results in second gas sensor data delivered to the controller 604 (see box 901 of FIG. 9). The controller 604 compares the second gas sensor data with a second gas calibration data set according to instructions for determining a metric on the processing in order to produce a second metric (see box 902 of FIG. 9). (The second target gas, second gas calibration data set, and instructions for determining the second metric may be the same as the first, or they may be different, according to various embodiments. Similarly, the chosen method of comparison between the second target gas sensor data and the second gas calibration data set may be any of the comparisons illustrated in FIGS. 7A-7F, including the same method of comparison chosen for the first gas sensor data and the first gas calibration data set, or it may be another method suitable for the chosen metric, according to various embodiments.) The controller 604 may then halt the processing based on the second metric, according to instructions for generating any necessary control signals. In some embodiments, the controller may make a binary choice either to terminate processing of the substrate or to return to monitoring the first target gas, corresponding to a return from box 903 of FIG. 9 to box 802 of FIG. 8. In other embodiments, monitoring the second target gas may continue, or there may be additional target gases or gas sensors to query for data to process and upon which to condition further decisions about process control.

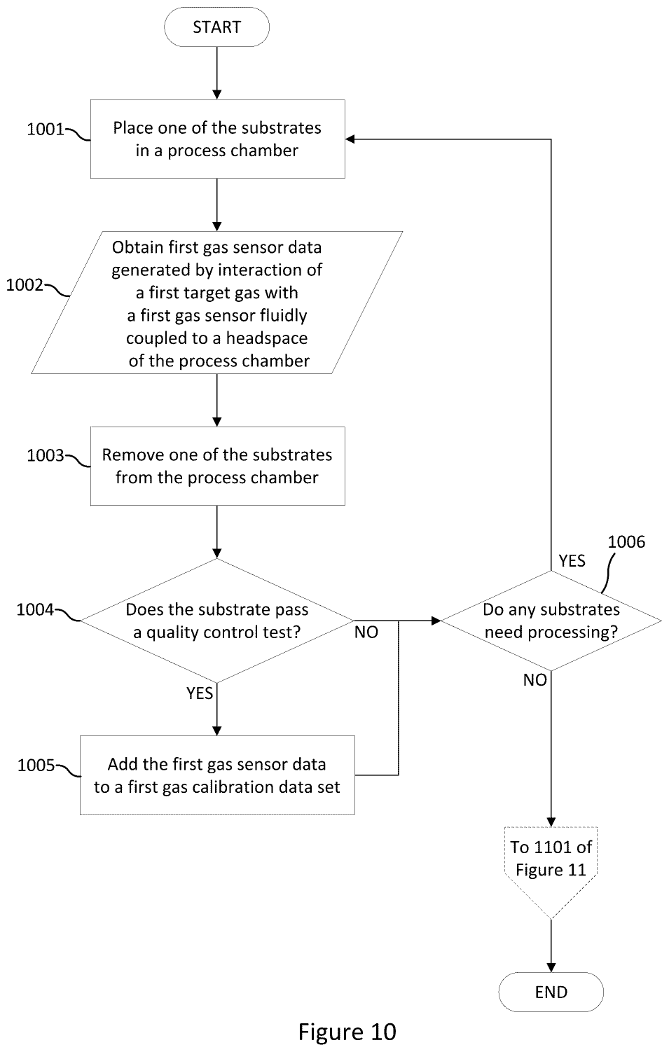

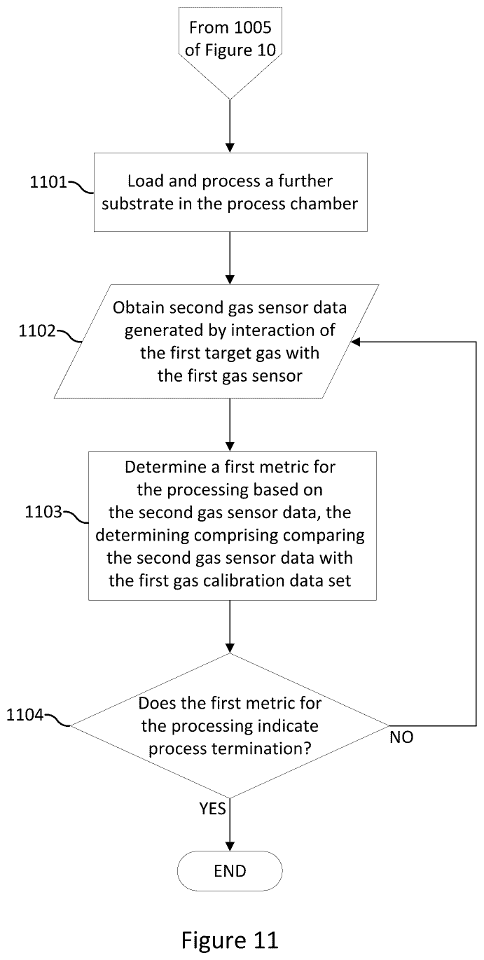

FIG. 10 provides a flow chart for a method of processing substrates, in which a cyclic measurement process applied to a plurality of substrates may be used to assemble a gas calibration data set, according to various embodiments. The method begins with loading one of the substrates into a process chamber, such as any of the process chambers depicted in FIGS. 1-5 (see Box 1001 of FIG. 10). Processing of the substrate and other physical phenomena that may occur while the substrate is present in the chamber may produce a first target gas that interacts with a first gas sensor fluidly coupled to a headspace of the process chamber, which interaction results in first gas sensor data delivered to the controller 604 (see box 1002 of FIG. 10). The substrate may then be removed from the process chamber (see box 1003 of FIG. 10) and subjected to quality control testing, such as optical inspection of the substrate or testing of individual dies from the wafer for function, according to various embodiments. If the wafer passes quality control testing, the first gas sensor data may be added to a first gas calibration data set (see box 1005 of FIG. 10), which may ultimately consist of a plurality of gas sensor data from passing substrates (in some embodiments) or to data derived therefrom in a manner suitable for the process in question, such as by taking a smoothed average (in other embodiments). A new cycle of the measurement process from box 1001 to box 1004 (and possibly 1005) of FIG. 10 may then begin, according to whether any substrates remain (see box 1006 of FIG. 10). Optionally, after all substrates provided for assembly of the first gas calibration data set have been exhausted, measurements on a further substrate may be made before halting, according to the method illustrated in FIG. 11.

FIG. 11 provides a flow chart for a method of processing a substrate after applying the method of FIG. 10. The method begins with loading a further substrate into the same process chamber as was used to assemble the first gas calibration data set, (see Box 1101 of FIG. 11). Processing of the substrate and other physical phenomena that may occur while the substrate is present in the chamber may produce the first target gas, which interacts with the first gas sensor to generate second gas sensor data delivered to the controller 604 (see box 1102 of FIG. 11). The controller 604 compares the second gas sensor data with the first gas calibration data set (corresponding to gas calibration data set 610) according to instructions for determining a metric on the processing (corresponding to instructions 612) in order to produce a first metric (see box 1103 of FIG. 11). (The chosen method of comparison may be any of the comparisons illustrated in FIGS. 7A-7F, or it may be another method suitable for the chosen metric, according to various embodiments.) The controller 604 may then halt the processing based on the first metric, according to instructions for generating any necessary control signals (corresponding to instructions 614). In some embodiments, the controller may make a binary choice either to terminate processing of the substrate or to continue processing while monitoring the first target gas, corresponding to a simple loop over boxes 1102-1104 of FIG. 11.

Example embodiments of the invention are described below. Other embodiments can also be understood from the entirety of the specification as well as the claims filed herein.

Example 1. A method of processing a substrate includes loading and processing the substrate in a process chamber, and obtaining first gas sensor data generated by interaction of a first target gas with a first gas sensor fluidly coupled to a headspace of the process chamber. The method includes determining a first metric for the processing based on the first gas sensor data, the determining including comparing the first gas sensor data with a first gas calibration data set. The method includes terminating the processing of the substrate based on the first metric for the processing.

Example 2. The method of example 1, where loading the substrate includes immersing a plurality of substrates in a bath solution contained in a batch tank of the process chamber, the substrate being part of the plurality of substrates.

Example 3. The method of one of examples 1 or 2, where the process chamber is connected to a gas inlet, and the first gas sensor is disposed in the gas inlet.

Example 4. The method of one of examples 1 to 3, where the process chamber is connected to a gas outlet, and the first gas sensor is disposed in the gas outlet.

Example 5. The method of one of examples 1 to 4, where a nozzle and a wafer support are disposed within the process chamber, the nozzle is configured to dispense material onto the wafer support, and the first gas sensor is disposed proximate to the nozzle.

Example 6. The method of one of examples 1 to 5, further including a dispensing arm, the first gas sensor being disposed on the dispensing arm.

Example 7. The method of one of examples 1 to 6, where the process chamber is connected to a drain system including a catch tank, and the first gas sensor is disposed in the catch tank.

Example 8. The method of one of examples 1 to 7, where the process chamber includes a hot plate for heating the substrate disposed on the wafer support.

Example 9. The method of one of examples 1 to 8, where the process chamber is part of a single-wafer processing tool.

Example 10. The method of one of examples 1 to 9, where the process chamber is part of a supercritical dryer.

Example 11. The method of one of examples 1 to 10, further includes obtaining second gas sensor data generated by interaction of a second target gas with a second gas sensor fluidly coupled to the headspace of the process chamber; and determining a second metric for the processing based on the second gas sensor data, the determining including comparing the second gas sensor data with a second gas calibration data set, where terminating the processing of the substrate is further based on the second metric for the processing.

Example 12. The method of one of examples 1 to 11, where the second target gas is identical to the first target gas.

Example 13. An apparatus includes a process chamber, a wafer support disposed in the process chamber, a headspace above the wafer support, and a gas sensor fluidly coupled to the headspace and configured to generate gas sensor data.

Example 14. The apparatus of example 13, further including: a memory; and one or more controllers coupled to the memory, the memory storing a first gas calibration data set and instructions to determine a first metric based on first gas sensor data obtained during processing of a substrate in the process chamber, the determining including comparing the first gas sensor data with the first gas calibration data set, and generate control signals to terminate the processing of the substrate based on the first metric for the processing.

Example 15. The apparatus of one of examples 13 or 14, where the process chamber is connected to a gas inlet, and the gas sensor is disposed in the gas inlet.

Example 16. The apparatus of one of examples 13 to 15, where the process chamber is connected to a gas outlet, and the gas sensor is disposed in the gas outlet.

Example 17. The apparatus of one of examples 13 to 16, where a nozzle and a wafer support are disposed within the process chamber, the nozzle is configured to dispense material toward the wafer support, and the gas sensor is disposed proximate to the nozzle.

Example 18. The apparatus of one of examples 13 to 17, where a dispensing arm supports the nozzle, and the gas sensor is disposed on the dispensing arm.

Example 19. The apparatus of one of examples 13 to 18, where the process chamber is connected to a drain system including a catch tank, and where the gas sensor is disposed in the catch tank.

Example 20. The apparatus of one of examples 13 to 19, where the process chamber includes a hot plate for heating a substrate disposed on the wafer support.

Example 21. The apparatus of one of examples 13 to 20, where the process chamber is part of a single-wafer processing tool.

Example 22. The apparatus of one of examples 13 to 21, where the process chamber is part of a supercritical dryer.

Example 23. A method of processing substrates includes performing a cyclic measurement process to generate a first gas calibration data set from the substrates. One cycle of the cyclic measurement process including: placing one of the substrates in a process chamber, obtaining first gas sensor data generated by interaction of a first target gas with a first gas sensor fluidly coupled to a headspace of the process chamber, removing the substrate from the process chamber, and performing a quality control test on the substrate and based thereon adding the first gas sensor data to a first gas calibration data set.

Example 24. The method of example 23, further including: loading and processing a further substrate in the process chamber; obtaining second gas sensor data generated by interaction of the first target gas with the first gas sensor; determining a first metric for the processing based on the second gas sensor data, the determining including comparing the second gas sensor data with the first gas calibration data set; and terminating the processing of the further substrate based on the first metric for the processing.

Example 25. The method of one of examples 23 or 24, where the process chamber is part of a single-wafer processing tool.

While this invention has been described with reference to illustrative embodiments, this description is not intended to be construed in a limiting sense. Various modifications and combinations of the illustrative embodiments, as well as other embodiments of the invention, will be apparent to persons skilled in the art upon reference to the description. It is therefore intended that the appended claims encompass any such modifications or embodiments.

What is claimed is:

1. A method of processing a substrate, the method comprising:
loading and processing the substrate in a process chamber;
obtaining first gas sensor data generated by interaction of a first target gas with a first gas sensor fluidly coupled to a headspace of the process chamber;
determining a first metric for the processing based on the first gas sensor data, the determining comprising comparing the first gas sensor data with a first gas calibration data set; and
terminating the processing of the substrate based on the first metric for the processing.

2. The method of claim 1, wherein loading the substrate comprises immersing a plurality of substrates in a bath solution contained in a batch tank of the process chamber, the substrate being part of the plurality of substrates.

3. The method of claim 1, wherein the process chamber is connected to a gas outlet, and the first gas sensor is disposed in the gas outlet.

4. The method of claim 1, wherein a nozzle and a wafer support are disposed within the process chamber, the nozzle is configured to dispense material onto the wafer support, and the first gas sensor is disposed proximate to the nozzle.

5. The method of claim 1, wherein the process chamber is connected to a drain system comprising a catch tank, and the first gas sensor is disposed in the catch tank.

6. The method of claim 1, wherein the process chamber comprises a hot plate for heating the substrate disposed on the wafer support.

7. The method of claim 1, wherein the process chamber is part of a single-wafer processing tool.

8. The method of claim 1, wherein the process chamber is part of a supercritical dryer.

9. The method of claim 1, further comprising:
obtaining second gas sensor data generated by interaction of a second target gas with a second gas sensor fluidly coupled to the headspace of the process chamber; and
determining a second metric for the processing based on the second gas sensor data, the determining comprising comparing the second gas sensor data with a second gas calibration data set, wherein terminating the processing of the substrate is further based on the second metric for the processing.

10. The method of claim 9, wherein the second target gas is identical to the first target gas.

11. An apparatus comprising:
a process chamber;
a wafer support disposed in the process chamber;
a headspace above the wafer support; and
a gas sensor fluidly coupled to the headspace and configured to generate gas sensor data.

12. The apparatus of claim 11, further comprising:
a memory; and
one or more controllers coupled to the memory, the memory storing a first gas calibration data set and instructions to
determine a first metric based on first gas sensor data obtained during processing of a substrate in the process chamber, the determining comprising comparing the first gas sensor data with the first gas calibration data set, and
generate control signals to terminate the processing of the substrate based on the first metric for the processing.

13. The apparatus of claim 11, wherein the process chamber is connected to a gas outlet, and the gas sensor is disposed in the gas outlet.

14. The apparatus of claim 11, wherein a nozzle and a wafer support are disposed within the process chamber, the nozzle is configured to dispense material toward the wafer support, and the gas sensor is disposed proximate to the nozzle.

15. The apparatus of claim 11, wherein the process chamber is connected to a drain system comprising a catch tank, and wherein the gas sensor is disposed in the catch tank.

16. The apparatus of claim 11, wherein the process chamber is part of a single-wafer processing tool.

17. The apparatus of claim 11, wherein the process chamber is part of a supercritical dryer.

18. A method of processing substrates, the method comprising:
performing a cyclic measurement process to generate a first gas calibration data set from the substrates, a cycle of the cyclic measurement process comprising:
placing one of the substrates in a process chamber,
obtaining first gas sensor data generated by interaction of a first target gas with a first gas sensor fluidly coupled to a headspace of the process chamber,
removing the substrate from the process chamber, and
performing a quality control test on the substrate and based thereon adding the first gas sensor data to a first gas calibration data set.

19. The method of claim 18, further comprising:
loading and processing a further substrate in the process chamber;
obtaining second gas sensor data generated by interaction of the first target gas with the first gas sensor;
determining a first metric for the processing based on the second gas sensor data, the determining comprising comparing the second gas sensor data with the first gas calibration data set; and
terminating the processing of the further substrate based on the first metric for the processing.

20. The method of claim 18, wherein the process chamber is part of a single-wafer processing tool.
